(19) United States
(12) Patent Application Publication
(10) Pub. No.: US 2026/0229323 A1
(43) Pub. Date: Aug. 6, 2026

(54) SYSTEM AND METHOD FOR GENERATING, STORING, AND SECURELY SHARING SMART PATIENT INSTRUCTIONS

(71) Applicants: Tarek A. Shazly, Pittsburgh, PA (US); Sherif A. Shazly, Leeds (GB)

(72) Inventors: Tarek A. Shazly, Pittsburgh, PA (US); Sherif A. Shazly, Leeds (GB)

(21) Appl. No.: 19/149,179

(22) PCT Filed: Jan. 19, 2024
(86) PCT No.: PCT/US2024/012172

§ 371 Date: Jul. 18, 2025

Related U.S. Application Data
(60) Provisional application No. 63/440,236, filed on Jan. 20, 2023.

(51) Int. Cl. G16H 10/60; G16H 80/00

(52) U.S. Cl. CPC G16H 10/60; G16H 80/00

(57) ABSTRACT

A system is disclosed for generating patient-related medical information. The system includes a healthcare provider transformer application that automatically converts patient medical information into a language suitable for sharing with various individuals involved in a patient's care. The healthcare transformer application receives medical data inputted by a healthcare provider and identifies key medical terms and abbreviations from the inputted data. It then translates these key terms and abbreviations into pre-defined simple language definitions and generates a legible simple text by combining the definitions. The system further hybridizes the simple text by adding extra separators, formatting, additional text, general advice, disclaimer, date and time, logos, images, illustrations, tables, and machine-readable codes such as barcodes, QR codes, and encrypted codes. The system generates a hybrid output based on the hybridized text, which includes instructions that are both human readable and machine-readable.

### CROSS-REFERENCE TO RELATED APPLICATION(S)

This application claims priority to, and the benefit of, co-pending U.S. Provisional Application No. 63/440,236, filed Jan. 20, 2023, for all subject matter common to both applications. The disclosure of said provisional application is hereby incorporated by reference in its entirety.

### FIELD OF THE INVENTION

The present invention relates to the field of healthcare, and more specifically to a system and method for generating, processing, storing and securely sharing easily usable smart patient-related medical information.

### BACKGROUND

Healthcare records are complex scripts that contain a plethora of medical terms, expressions, standardized and non-standardized medical abbreviations that make the language of such scripts difficult for the patient to understand. Globally, healthcare systems have been encouraging their patients to actively participate in their care. One important aspect of this movement is granting instant access of patients to their health records through web portals and smartphone apps. One of the challenges to this approach is the complexity of encounter notes that are written with the priority of preserving medical information in a scientific way rather than for sharing with patients. Furthermore, sharing these notes with other healthcare providers involved in patient care can be critical. Medical notes written by a healthcare provider of a subspecialized field of medicine can be as complex to interpret by a specialist of a different field of medicine or a general practitioner. The state-of-the-art solution to this issue has been via utilizing letters which are derivatives of the standard encounter note with various levels of technical details and length depending on the recipient. The process has not changed in centuries for handwritten, typed, dictated, and electronic letters as the provider have to generate the original note, then generate all other needed documents manually such as a letter to the referring specialist, primary care physician, patient, and family. Sometimes certain segments of the provider notes need to be transformed from their “medical format” to the issued patient medical instructions or to explain certain parts of their diagnoses list or medical plan in plain language.

“SOAP notes” are a standard format for documenting patient information in a healthcare setting. They are structured around four main elements: Subjective, Objective, Assessment, and Plan. The subjective portion includes the patient's symptoms and complaints, medical history, and personal/social history. The objective portion includes observed and measurable information such as vital signs, exam findings, and test results. The assessment portion includes the healthcare provider's interpretation of the patient's information and may include a diagnosis or differential diagnosis. The plan portion includes treatment and follow-up recommendations and medical patient instructions. SOAP notes are useful for tracking progress and informing patient care.

SOAP note's structure varies depending on the specialty or the sub-specialty of the health care provider and other factors such as if the note is for in-patient or clinic setting encounters. For example, an outpatient eye visit SOAP note would include information specific to the eye exam. The subjective portion may include information about the patient's vision and any eye-related symptoms they are experiencing. The objective portion may include information gathered during the eye exam, such as visual acuity, eye movements, and pupil reactions. The assessment portion may include a diagnosis or differential diagnosis of any eye conditions or problems identified during the exam. The plan portion may include recommendations for treatment with eye drops or follow-up care, such as prescribing glasses or referring the patient to a specialist.

It is important for patients to properly understand and follow medical instructions to effectively manage their health and achieve the best possible outcomes from the provided healthcare. Proper understanding and adherence to medical instructions can help to prevent complications and ensure that treatment is effective. For example, if a patient does not take their medication as prescribed, they may not receive the full benefits of the treatment, or they may experience negative side effects due to improper dosing. Similarly, if a patient does not follow instructions for wound care or physical therapy exercises, they may experience delays in healing or reduced effectiveness of the treatment. Proper understanding and adherence to medical instructions can also help to prevent the spread of infection and the development of chronic conditions. Overall, following medical instructions is crucial for achieving and maintaining good health.

Medical instructions refer to specific guidance provided by healthcare professionals on how to care for one's health, including taking medications as prescribed, caring for wounds, performing physical therapy exercises, managing chronic conditions, following up with healthcare providers, making lifestyle changes, and handling emergencies.

Patient adherence to prescribed medical treatment, including taking medications as directed, is important for successful outcomes. However, patients may have difficulty understanding or following complex instructions, especially if they are elderly, have cognitive decline, or are taking multiple medications. Using plain language and illustrated instructions can help patients, especially the elderly, to understand and follow their treatment plans, which can be especially important in preventing medication errors. By simplifying communication and empowering elderly patients to manage their own health, healthcare providers can improve patient quality of life.

smart pill holders are one mechanism designed to help patients optimize their medication use and log their adherence to medications. They can be programmed to send reminders to a patient's smartphone or tablet at the appropriate times to take medication and can also dispense medication at the appropriate time. The device can also be programmed to log the patient's medication use and send this information to their healthcare provider or other designated parties to help identify issues with medication adherence and adjust the patient's treatment plan as needed. However, the complexity of programming the device may pose a limitation for elderly patients and there is no direct way to communicate medical instructions from healthcare providers into the device, which leaves room for misinterpretation or incorrect data entry by patients or their caregivers. Additionally, the time-consuming nature of programming the device may make it impractical for healthcare providers to do so for their patients.

Furthermore, managing non-pill medications presents its own challenges. For example, managing eye drop medication can be especially challenging for patients, as unlike oral medications, The eye medication is typically stored in a single bottle or tube that contains a large number of doses that are intended to be taken over a period of several weeks or months. The bottle or tube serves as one physical unit that cannot be physically divided and placed into different pill holder compartments until the time of use. It is typically the case that the patient has one bottle or tube at a time of the eye drops or ointment that they use as they last for weeks or months. This makes such medication holders useless for eye drops and ointments.

Overall, managing eye drop medication can be a challenging task for patients, and it is important for healthcare providers to provide clear and easy-to-follow instructions to help patients manage their medication effectively. This may include providing written instructions, using visual aids, and allowing extra time for patient education and questions.

### SUMMARY

There is a need for a system and method that can streamline and partially or completely automate the process of generating different versions of the medical information to properly match the recipients with whom it is shared. This is especially needed to generate easily usable instructions for patients that are safe, effective, and efficient, to help them properly follow their medical instructions to achieve the best possible medical outcomes.

The present invention provides a system and method for generating easily usable smart patient-related medical information. The system comprises a healthcare provider transformer application that automatically converts patient medical information in a way that is customizable by the healthcare provider. The transforming software processes the patient-related medical information locally, using a local transformation processing, local artificial intelligence engine, or sends the information after pre-processing to an internet server for further processing to produce easy to understand, reader-appropriate medical information. The generated information is suitable for storage, sharing with other healthcare providers, ancillary personnel and staff involved in patient care, patients, and their caretakers. Additionally, the transformer software can generate instructions from standard medical format into hybrid output that is both human and machine-readable. The transformer software can additionally hash the newly generated information and/or instructions into the blockchain and/or mint non-fungible tokens (NFTs) that contain the instructions and include access to them in the blockchain. The hybrid output includes simple instructions with clear text with or without tables and/or illustrations or clarifications, as well as machine-readable elements such as barcodes, QR codes, encrypted codes, and customizable website/application links that allows interactions with other software, machines or allow access to the blockchain or the data stored online. The system can also include a user software application that can read and process the hybrid output and generate additional patient instructions in different forms and customize alarms, calendar events, reminders, patient compliance logs, and means to communicate with the healthcare provider. The system may also include a dedicated website that can process the data in the hybrid output to further display patient instructions, set reminders, or calendar event invitations through the browsing device operating system and interact with a smart device to set physical device reminders/usage data. The patient may directly or indirectly interact with the hybrid output, the native user application, the dedicated website, or the smart device to properly follow medical instructions, medications or eye drop use and log patient usage. Furthermore, the application and/or the website can gamify the usage of medications such as via rewarding the patient points, stars, and rank usage.

In accordance with example embodiments of the present invention, a system for generating patient-related medical information is provided. The system includes a healthcare provider transformer application for automatically converting patient medical information into a language suitable for sharing with a variety of individuals involved in a patient's care in a way customizable by the healthcare provider. The healthcare transformer application configured to: receive medical data inputted by a healthcare provider; identify key medical terms and abbreviations from the inputted data; translate the key medical terms and abbreviations into pre-defined simple language definitions; generate a legible simple text by combining the definitions; hybridize the simple text by adding one or more of extra separators, formatting, additional text, general advice, disclaimer, date and time, logos, images, illustrations, tables, and a machine-readable barcode, QR code, and encrypted code; generate an hybrid output based on the hybridized text; and output the hybrid output wherein the hybrid output comprises instruction that are both human readable and machine-readable.

In accordance with aspects of the present invention, the transformer application processes the patient-related medical information locally, using a local transformation processing, local artificial intelligence engine, or sending the information after pre-processing to an internet server for further processing to produce easy to understand, reader-appropriate medical information.

In accordance with aspects of the present invention, the hybrid output further comprises a plan.

In accordance with aspects of the present invention, the transformer application hashes generated information and/or instructions into a blockchain and/or mints non-fungible tokens (NFTs) that contain the instructions and include access to the instructions in the blockchain.

In accordance with aspects of the present invention, the healthcare transformer application is further configured to: optimize the provider transformer software to match needs of the healthcare provider, including setting preferences for local plain text processing, local AI processing, or internet AI processing; select specialty dictionaries for the transformer software; create templates for different ways of processing medical information; de-identify data, optimize results of AI processing, or use a local customizable AI software module to perform tasks in an automated or semi-automated way; and transmit customization parameters combined with raw or processed patient medical information to an AI software via secure ways.

In accordance with aspects of the present invention, the system further comprises a user software application for reading and processing the hybrid output and generating additional patient instructions in different forms and customizing alarms, calendar events, reminders, patient compliance logs, and means to communicate with the healthcare provider the user software application configured to: receive the hybrid output; extract and interpret relevant information from the instructions of the hybrid output; present the instructions to the patient in a clear and easy-to-understand format; and set reminders and alarms to help the patient follow the instructions.

In accordance with aspects of the present invention, the system further comprises a dedicated website for processing data in the hybrid output to further display patient instructions, set reminders, or calendar event invitations through a browsing device operating system and interact with a smart device to set physical device reminders/usage data, the dedicated website configured to: receive hybrid output; extract and interpret relevant information from the instructions of the hybrid output; present the instructions to the patient in a clear and easy-to-understand format; and set reminders and alarms to help the patient follow the instructions.

In accordance with aspects of the present invention, a patient may directly or indirectly interact with the hybrid output, a user application, a dedicated website, or a smart device to properly follow medical instructions, medications or eye drop use and log patient usage. In some such aspects, the application and/or the website gamifies the usage of the medications such as via rewarding patient points, stars, and rank usage.

In accordance with example embodiments of the present invention, a system for generating patient instructions from medical instructions is provide. The system includes a healthcare provider transformer application on a healthcare computing device that receives medical instructions from a healthcare provider in a medical format and converts the medical instructions into an hybrid output; a user software application on a user computing device that processes the hybrid output to generate additional patient instructions in different forms; a dedicated website that enables access to the hybrid output; and a smart device that interacts with the user software application or the dedicated website to set physical device reminders and usage data. The hybrid output includes layman instructions with clear text and/or tables and/or illustrations, and machine-readable features such as barcodes, QR codes, encrypted codes, and customizable website links. The hybrid output is screen displayable, printable, and electronically transferable. The user software application is configured to generate large, high contrast text or text-to-speech output for users with visual impairments, customize alarms and reminders, and track patient compliance on reading the hybrid output.

In accordance with aspects of the present invention, the healthcare provider transformer application includes text identification that identifies a style of medical writing, and a text translation step that converts medication names into an appropriate name, correcting typos, replacing abbreviations with full names, and translating frequency of use.

In accordance with aspects of the present invention, the healthcare provider transformer application includes text generation that combines different pieces of medication name, frequency, mode of use, etc. into legible sentences, and text hybridization step that adds extra separators, formatting, additional text such as information about the medications, logos, images, illustrations, tables, and the machine-readable barcode, QR code, encrypted code.

In accordance with aspects of the present invention, the healthcare provider transformer application includes output generation that is adjustable by the healthcare provider to choose the output format and what is included in the output generated.

In accordance with aspects of the present invention, the healthcare provider transformer application is further configured to decode an impression and plan section of a patient chart.

In accordance with example embodiments of the present invention, a method for transforming medical data is provided. The method includes: inputting medical data by a healthcare provider into a provider transformer software; identifying key medical terms and abbreviations from the inputted data; translating the key medical terms and abbreviations into pre-defined simple language definitions; generating a legible simple text by combining the definitions; hybridizing the simple text by adding extra separators, formatting, additional text, general advice, disclaimer, date and time, logos, images, illustrations, tables, and a machine-readable barcode, QR code, or encrypted code; generating a hybrid output based on the hybridized text; and outputting the hybrid output.

In accordance with aspects of the present invention, the method further includes: optimizing the provider transformer software to match needs of the healthcare provider, including setting preferences for local plain text processing, local AI processing, or internet AI processing; selecting specialty dictionaries for the transformer software; creating templates for different ways of processing medical information; de-identifying data, optimizing results of AI processing, or using a local customizable AI software module to perform tasks in an automated or semi-automated way; and transmitting customization parameters combined with raw or processed patient medical information to an AI software via secure ways.

In accordance with example embodiments of the present invention, a user software application for providing patient instructions is provided. The user software application includes a processor configured to receive hybrid patient instructions and generate additional patient instructions in different formats; a user interface configured to present patient instructions in a clear and easy-to-understand format; and a reminder module configured to set reminders and alarms for patient compliance.

In accordance with example embodiments of the present invention, a method for providing patient instructions is provided. The method involves: receiving hybrid patient instructions; extracting and interpreting relevant information from the instructions; presenting the instructions to the patient in a clear and easy-to-understand format; and setting reminders and alarms to help the patient follow the instructions.

In accordance with example embodiments of the present invention, a method for processing hybrid patient instructions is provided. The method involves: a) receiving hybrid patient instructions, via camera scanning of hybrid output elements, such as a QR code, or manually entered by the patient, or transmitted from a provider transformer application; b) extracting and interpreting relevant information from the hybrid patient instructions, including prescribed medications and dosages, frequency and method of administration, and any relevant reference materials or patient education resources; c) presenting the instructions to the patient in a clear and easy-to-understand format, using text, tables, illustrations, or other visual aids as needed; d) setting reminders and alarms, alerting the patient when it is time to take their medications, triggered based on the time of day, the patient's location, or other factors, and delivered via the application's user interface or via external means such as push notifications or emails; e) tracking the patient's medication usage and adherence, via a log, which may be automatically populated based on the patient's input, or manually entered by the patient; f) utilizing gamification techniques to motivate the patient to take their medications as prescribed; g) providing personalized feedback to users based on their medication usage patterns; h) offering virtual rewards or incentives for medication usage; i) connecting users with others who are also using medications; j) awarding points or other virtual rewards for medication usage; k) tracking users' progress towards their medication usage goals; l) including a leaderboard or other social comparison feature; and m) incorporating gamified education content.

In accordance with example embodiments of the present invention, a system for providing healthcare instructions is disclosed, the system includes: a healthcare provider transformer application configured to receive a medical instruction and generate a hybrid output; a user software application configured to receive the hybrid output generate instructions based on the hybrid output; a dedicated website configured to allow a healthcare provider to customize the hybrid output; and a smart device configured to store the hybrid output and transmit the hybrid output to a user.

In accordance with aspects of the present invention, the healthcare provider transformer application is further configured to receive patient information and generate a medical instruction based on the patient information.

In accordance with aspects of the present invention, the user software application is further configured to receive the hybrid output from the healthcare provider transformer application and generate a user interface for displaying the hybrid output.

In accordance with aspects of the present invention, the smart device is further configured to track the user's medication usage and adherence over time.

In accordance with aspects of the present invention the smart device includes means to sense its contents via weight, color, and computer vision.

In accordance with aspects of the present invention, the smart device includes different illumination colors for each compartment and a display that provides the user with instructions, reminders, and other information.

In accordance with aspects of the present invention, the smart device includes notification methods such as sound, light, vibration, and messages on a display to signal reminders and messages to the user.

In accordance with example embodiments of the present invention, a system for providing smart hybrid patient instructions is disclosed. The system includes: a user software application configured to receive hybrid patient instructions and generate additional patient instructions in different formats; a dedicated website configured to process machine-readable data on the patient instructions; and a smart device configured to produce physical device reminders and collect usage data based on interactions with the patient instructions.

In accordance with example embodiments of the present invention, a method for providing healthcare instructions is disclosed. The method involves: receiving a medical instruction from a healthcare provider; generating an hybrid output based on the medical instruction; customizing the hybrid output; storing the hybrid output on a smart device; and transmitting the hybrid output to a user.

In accordance with aspects of the present invention, the method further involves receiving patient information and generating the medical instruction based on the patient information.

In accordance with aspects of the present invention, the method further involves generating a user interface for displaying the hybrid output.

In accordance with aspects of the present invention, the method further involves tracking the user's medication usage and adherence over time.

In accordance with aspects of the present invention, the method further involves sensing contents of the smart device via weight, color, and computer vision.

In accordance with aspects of the present invention, the method further involves providing different illumination colors for each compartment and a display that provides the user with instructions, reminders, and other information.

In accordance with aspects of the present invention, the method further involves providing notification methods such as sound, light, vibration, and messages on a display to signal reminders and messages to the user.

In accordance with example embodiments of the present invention, a method for transforming medical information into a language suitable for sharing with a variety of individuals involved in a patient's care is provided. The method involves: obtaining/receiving medical information; pre-processing the medical information to replace certain abbreviations, terms, or structures; utilizing a local AI with NLP engine to process the medical information; and reformatting the processed medical information and displaying it to a user for approval before sharing and/or saving the processed medical information.

In accordance with example embodiments of the present invention, a system for transforming medical information into a language suitable for sharing with a variety of individuals involved in a patient's care is provided. The system includes: a transformer application configured to pre-process the medical information to replace certain abbreviations, terms, or structures; a local AI with NLP engine configured to process the medical information; and a display configured to reformat an output and display it to a user for approval before sharing and/or saving the processed medical information.

In accordance with example embodiments of the present invention, a computer program product for transforming medical information into a language suitable for sharing with a variety of individuals involved in a patient's care is provided. The computer program product comprises: a computer-readable storage medium having instructions stored thereon that, when accessed by a patient software, cause the software to: pre-process the medical information to replace certain abbreviations, terms, or structures; utilize a local AI with NLP engine to process the medical information; and reformat an output and display it to a user for approval before sharing and/or saving the processed medical information.

In accordance with example embodiments of the present invention, a method for transforming medical information into a language suitable for sharing with a variety of individuals involved in a patient's care is provided. The method involves: de-identifying patient medical data in a note in a HIPAA-compliant manner; selecting pertinent parts of the medical information to submit to an internet server; standardizing any non-standardized or specialized abbreviations in text in a customizable way; attaching customization parameters along with processed medical information to submit to AI software on an internet server; receiving back the medical information processed by the internet AI server; and reformatting an output and displaying the processed medical information to a user for approval before sharing and/or saving the processed medical information.

In accordance with example embodiments of the present invention, a system for transforming medical information into a language suitable for sharing with a variety of individuals involved in a patient's care is provided. The system includes: a transformer application configured to de-identify patient medical data in a note in a HIPAA-compliant manner; a processor configured to select pertinent parts of the medical information to submit to an internet server and standardize any non-standardized or specialized abbreviations in text in a customizable way; an AI software on the internet server configured to receive processed medical information and customization parameters; and a display configured to reformat an output and display the processed medical information and customization parameters to a user for approval before sharing and/or saving the processed medical information.

In accordance with example embodiments of the present invention, a computer program product for transforming medical information into a language suitable for sharing with a variety of individuals involved in a patient's care is provided. The computer program product comprises: a computer-readable storage medium having instructions stored thereon that, when executed by a processor, cause the processor to: de-identify patient medical data in a note in a HIPAA-compliant manner; select pertinent parts of the medical information to submit to an internet server; standardize any non-standardized or specialized abbreviations in the medical information in a customizable way; attach customization parameters along with the medical information to submit to an AI software on the internet server; receive back the medical information processed by the internet AI server; and reformat an output and display the processed medical information to a user for approval before sharing and/or saving the processed medical information.

In accordance with example embodiments of the present invention, a method for improving medical instructions for patients is provided. The method involves: inputting patient medical information into a decentralized blockchain network; and encoding the information as a non-fungible token (NFT); and providing a hybrid output, native application, dedicated website, or smart device to enable patient adherence to medical instructions.

In accordance with example embodiments of the present invention, a system for hashing easily usable smart patient instructions onto a blockchain is provided. The system includes: a healthcare provider transformer application for receiving patient medical instructions in their original format from a healthcare provider; a blockchain for hashing the instructions and storing the hashed instructions; the healthcare provider transformer application for processing the medical instructions into a hybrid output, the hybrid output being both patient and machine-readable; and the healthcare provider transformer application for generating an access code and adding the access code to the hybrid output; and the patient accessing the hashed instructions on the blockchain using the access code provided in the hybrid output.

In accordance with aspects of the present invention, the hybrid output includes layman instructions that are easily understandable by a patient and machine-readable features such as barcodes, QR codes, encrypted codes, and customizable website links.

In accordance with aspects of the present invention, the patient has an option of generating a non-fungible token (NFT) from the hashed instructions. In some such aspects, a NFT is generated from the hashed instructions and stored on the blockchain, providing an additional layer of security and verifiability for the instructions. In further aspects the healthcare provider transformer application adds an access code, links and/or thumbnails to the hashed instructions and/or NFT and includes it in the hybrid output. In some such aspects, the access code allows the patient to access the hashed instructions on the blockchain.

In accordance with example embodiments of the present invention, a method for hashing easily usable smart patient instructions onto a blockchain is provided. The method involves: receiving patient medical instructions in their original format from a healthcare provider; processing the medical instructions into a hybrid output, the hybrid output being both patient and machine-readable; hashing the instructions and storing the hashed instructions on a blockchain; generating an access code and adding the access code to the hybrid output; and allowing the patient to access the hashed instructions on the blockchain using the access code provided in the hybrid output.

In accordance with aspects of the present invention, the hybrid output includes layman instructions that are easily understandable by a patient and machine-readable features such as barcodes, QR codes, encrypted codes, and customizable website links.

In accordance with aspects of the present invention, the patient has an option of generating a non-fungible token (NFT) from the hashed instructions. In some such aspects, the NFT is generated from the hashed instructions and stored on the blockchain, providing an additional layer of security and verifiability for the instructions. In further aspects, the method further includes adding an access code, links and/or thumbnails to the hashed instructions and/or NFT and including it in the hybrid output. In some such aspects, the access code allows the patient to access the hashed instructions on the blockchain.

The present invention discloses a system and method for generating easily usable versions of medical information and smart patient instructions. The system includes a healthcare provider transformer application that converts patient medical information in their medical format for example a complete medical (SOAP) note or some of its parts into different versions of the note or its parts suitable for sharing such as medical correspondence with other healthcare providers, consultation requests, referral notes, letters to patients, a patient viewable note, or medical peer to peer or prior authorization letters and patient instructions. The system can produce an hybrid output of instruction that is both patient and machine-readable. The hybrid output includes instructions in layperson terms with clear text, associated tables and/or illustrations, along with machine-readable features such as barcodes, QR codes, encrypted codes, and customizable website links. The machine-readable code is readable using a smart device such as a smartphone or tablet to generate text, graphic, audio and video output using its native camera application with or without assistance from dedicated applications.

The healthcare provider transformer application can assist healthcare providers in the processing and sharing of patient medical information with patients and members of their care team. This application allows healthcare providers to input patient medical information in a variety of ways, including typing, copy-pasting from electronic medical records, electronic medical records to transformer application direct communication, dictation, and automatically or semi-automatically converts this received information into a sharable output that can be customized according to the needs of the healthcare provider.

The healthcare provider transformer application uses common computational techniques such as parsing, replacement, translation, and re-formatting to process certain patient-related medical information locally on the healthcare provider's computer. This approach is ideal for the well-structured segments of the medical notes such as medication instructions and impression, plan, and patient instructions. This approach helps minimize AI guessing/probability errors to maintain patient confidentiality by avoiding the need to send the information to an internet server. In addition, the transformer application can use a local customizable artificial intelligence (AI) engine to assist in the processing of the information.

If the healthcare provider elects to utilize complex natural language processing power to be applied to the medical information in the notes, the transformer application can utilize a local AI with an NLP engine to process the medical information. The transformer application can pre-process the medical information to replace certain abbreviations, terms, or structures on the medical info before submitting it to the local AI engine and determine the parameters submitted to the local AI engine to optimize the output to match the desired audience determined by the user health care provider. Since the data does not leave the secure environment of the computer system/network within the healthcare system onto the internet, the transformer application may not necessarily de-identify the information unless deemed necessary.

When the healthcare provider elects to utilize even more complex natural language processing power to be applied to larger segments of the notes or complete medical notes for example to exchange communications, progress reports, or letters, the transformer application can utilize the big data internet AI and NLP servers. Before the patient medical information is transmitted to the internet AI servers, it is pre-processed by the transformer app to achieve certain goals. The provider transformer application processes the patient medical data in the note to de-identify it from identifying information such as names, contact information, providers names, social security numbers and many other pieces of information in a HIPAA-compliant manner to de-identify the patient. The process of de-identification performed by the provider transformer application is a multi-layer deidentification process involving a multi-step approach. Some steps involve obtaining additional information from the EMR or the provider to identify confidential information specifically related to the chart that needs de-identified. Additionally, the transformer application based on the provider's settings and intended recipient of the information to be generated will process the medical information. Such processing comprises selecting the pertinent parts of the medical information to submit to the internet server, standardize any non-standardized or specialized abbreviations in the text in a customizable way. For example, if the transformation is intended to share specialty or subspeciality SOAP notes with general practitioner or a specialist in a different field, specialty-specific abbreviations are translated into what they stand for but not translated into plain language and common medical terms are not translated. While if the medical information is meant for sharing with the patient, the transformer application translates all medical abbreviations and medical terms into their plain language definitions using a dictionary/map of terms and abbreviations. The healthcare provider application then attaches customization parameters along with the processed medical information to submit to the AI software on the internet server to use when processing the medical information. These customization parameters communicate with the internet AI algorithm certain parameters such as how short vs long, simple language vs formal, probability taking, creativity degree, which part of the note to focus more on, who is to be addressed (self, PCP, referred to physician) in a deidentified way. The health care provider can create preset options in the transformer application such as “letter to specialist,” which allows for the use of more detailed and technical language, “letter to primary care or general practitioner,” which is less technical and more focused on pertinent information for a non-specialist, “letter to patient” or “letter of medical justification” customizing the header description that accompany the text to the internet server via API (Application Programming Interface) or other secure protocols of submission of information.

The transformer application then receives back the medical information processed by the internet AI server, verifies it, reassembles it along with its original identifying information along with any other needed elements of text and or diagrams and reformats the output, and displays it to the user for approval before sharing and/or saving the processed medical information.

By eliminating the need for the health care provider to manually deidentify the medical information, enter medical information onto AI servers, and try multiple settings and customization parameters then manually correct misinterpreted terms and abbreviations. The system can improve the safety, accuracy, and efficiency of the transformation of medical information for sharing and saving. For example, an abbreviation like “PVR” may mean proliferative vitreoretinopathy (an eye problem) or peripheral vascular resistance (a blood flow parameter) depending on the specialty of the author of the note. This may create confusion and if the data was provided to the AI algorithm as “PVR” while less confusing if provided to the AI model as ‘proliferative vitreoretinopathy’ as translated by the transformer application during pre-processing as the transformer app is already optimized for the provider's abbreviations and needs.

The transformer application can also generate hybrid outputs, especially of specific segments of the patient note such as medication instructions, terms specifically used in patient notes, and other sections like allergies that are suitable for sharing with patients and their caretakers that are human and machine-readable. These outputs may be less technical and use more informal language and may include diagrams and figures to aid in understanding. The generated output can be shared with a variety of individuals involved in the patient's care, including other healthcare providers and ancillary personnel. The machine-readable elements of the hybrid output can either encode non-identifying information such as patient medication instructions, post-op care, or explanations of terms and abbreviations used in their notes without patient identifying information. Alternatively, password-encrypted information can be coded in the machine-readable elements of the hybrid outputs. Such hybrid outputs can include printed paper(s), an image on a screen, or an email.

In addition to simplifying and standardizing medical language, the healthcare provider transformer application also can improve the efficiency of the healthcare system by streamlining the sharing of patient medical information. This can help to reduce the time and resources needed to communicate and coordinate patient care, leading to better patient outcomes.

The system may also include means to further process the hybrid output such as patient/user software application that can generate additional patient instructions in different formats, additional explanations, references, illustrations and customized alarms or reminders, patient compliance logs, and means to communicate with the healthcare provider. The system may also include a dedicated website that can process the machine-readable data on the hybrid output to further provide additional patient instructions, set reminders through the browsing device operating system. The system may also include a smart dedicated device such as a smart medication or eye drop dispenser or holder that can produce physical device reminders and collect usage data based on its interactions with the smart patient instruction and/or the patient user software or instructions website.

The patient may interact with the hybrid output, the native application, the dedicated website, or the smart device to properly follow medical instructions for proper medications/eye drop use. This can improve patient adherence to medications and avoid obstacles such as forgetting to use the medications or using them incorrectly.

Additionally, the system and method can utilize blockchain technology and non-fungible tokens (NFTs) to improve medical instructions for patients. The system involves creating a digital record of the patient's medical instructions on a decentralized blockchain network and encoding this information as an NFT. The NFT serves as a secure and verifiable representation of the patient's medical instructions and can be stored on the blockchain or accessed through a QR code or similar means that are included in the hybrid output.

The system begins by inputting the patient's medical information in their more technical/medical form and/or the simple layman's terms into the blockchain network. This information may include their medication instructions, post-operative care instructions, allergies, emergency interventions in case of emergencies, and other critical details that are essential for the patient's care. The information is then hashed and added to the blockchain, creating a tamper-evident record of the patient's medical information.

The patient's medical instructions can also then be encoded in their text, audio, video, graphical, or QR code as an NFT using a smart contract on the blockchain. The NFT is a unique digital asset that represents ownership of the patient's medical information and can be stored on the blockchain or accessed through a link, alphanumeric code, QR code, or similar means. The NFT is non-fungible, meaning that it cannot be exchanged for other assets on a one-to-one basis, and it can be verified using the blockchain.

In the event of an emergency, the patient can present their hybrid output, NFT, either in physical form or via the QR code, to healthcare providers. The healthcare providers can then scan the QR code or access the NFT on the blockchain to retrieve the patient's medical instructions that can be urgently needed. This allows the healthcare providers to access the patient's medical instructions quickly and accurately, ensuring that the patient receives the appropriate care and interventions.

The smart patient instruction user application is a software program designed to assist patients in following their medical instructions and improving their adherence to medications. The application receives input in the form of hybrid patient instructions, which are issued by healthcare providers and contain both machine-readable and human-readable information.

Upon receiving the hybrid patient instructions, the patient application processes the input to extract and interpret the relevant information. This includes analyzing the instructions to identify the specific medications and dosages prescribed, as well as the frequency and method of administration. The application also parses the instructions to identify keywords and phrases, such as the names of medications, dosage amounts, and administration times. Any common typos or errors in the instructions may also be corrected during this process.

Once the relevant information has been extracted and interpreted, the application presents the instructions to the patient in a clear and easy-to-understand format. This may include text-based instructions, tables, illustrations, or other visual aids. The instructions may also be accompanied by reference materials or patient education resources, providing the patient with additional information on their medications and treatment.

To help the patient follow the instructions and take their medications as prescribed, the application includes features to set reminders and alarms. These reminders may be triggered based on the time of day, the patient's location, or other factors, and may be delivered via the application's user interface or via external means such as push notifications or emails. The application may also allow the patient to customize their reminders, setting them to repeat at regular intervals or adjusting their frequency and timing as needed.

To track the patient's medication usage and adherence, the application includes a log that records the dates and times that medications were taken. This log may be automatically populated based on the patient input, the camera capturing of the eye drops, scanning a drop bar or QR code as well as different methods by which the user confirms medication usage.

In the current invention, the challenge of the patient using the pill holder, and managing smart pill holder programming via allowing the entry of the healthcare provider-provided medical instructions into the pill holder using a machine-readable code such as QR code or encoded text directly or via a computer or smartphone application. This can help to eliminate the time-consuming and potentially dangerous task of data entry for the patient, as the healthcare provider can program the pill holder with the appropriate instructions and reminders.

For example, the healthcare provider could program the pill holder via a projected or printed encrypted text, bar or QR code with reminders to take the eye drops at specific times of the day, as well as with any necessary information about the dosage and frequency of the medication. The patient can then simply scan the QR code or input the encoded text into the pill holder directly or via an intermediary computer/smartphone to access the instructions and reminders. Unless the code is consistent with the format expected by the device or app, it will be rejected to avoid incorrect medical instruction entry.

By eliminating the need for patients to manually enter this information, an application and/or the smart pill holder that can be programmed by the healthcare provider can help to improve medication adherence and reduce the risk of errors. It can also help to streamline the process of managing eye drop medication and make it easier for patients to follow their treatment plans.

Software that generates simple and easy-to-follow patient medication instructions can benefit both healthcare providers and patients. For healthcare providers, such a tool can save valuable time by automating the process of creating medication instructions that already exist in health records, allowing them to focus on other important tasks. This can also improve patient adherence to medication, as clear and concise instructions can help patients better understand and follow their treatment regimen. Patient satisfaction is also likely to increase, as they will have a better understanding of their medications and how to take them correctly. Additionally, the risk of medication errors can be reduced, as the instructions will be clear and easy to follow. Finally, the load of patient messages and phone calls for clarification of medication instructions can be reduced as well, improving efficiency and productivity for healthcare providers. Overall, software that generates simple and easy-to-follow patient medication instructions can improve the quality of care and patient outcomes.

Overall, this system and method provides a secure, convenient, and efficient way for patients to manage and share their medical information with healthcare providers, improving the quality and effectiveness of patient care. It also has the potential to streamline healthcare processes and reduce the risk of errors by providing a tamper-evident record of the patient's medical information.

### BRIEF DESCRIPTION OF THE DRAWINGS

These and other characteristics of the present invention will be more fully understood by reference to the following detailed description in conjunction with the attached drawings, in which:

FIG. 1 is a block diagram illustrating the components of the system for generating easily usable smart patient instructions according to an embodiment of the present invention;

FIG. 2 is a flowchart of the processes by which the provider transformer software converts the provider input into the hybrid output;

FIG. 3 is a flowchart of the processes by which the provider transformer software converts the provider's instruction and plan into the hybrid output instruction and plan;

FIG. 4 is a flowchart of the processes by which the healthcare provider transformer application transformed medical info in the SOAP note into a sharable output that is customized to the needs of the healthcare provider;

FIG. 5 is a flowchart of the processes by which the user software 14 converts the machine-readable codes on the hybrid output into simple smart patient instruction and plan translation;

FIG. 6 is a flowchart illustrating the process of hashing the hybrid output onto a blockchain according to an embodiment of the present invention;

FIG. 7 is a flowchart illustrating the process of interaction between the user application and the hybrid output according to an embodiment of the present invention;

FIG. 8 is a flowchart illustrating the process by which the smart container interacts with the patient and hybrid instructions; and

FIG. 9 is a diagrammatic illustration of a computing device and related hardware for use in the implementation of the present invention.

### DETAILED DESCRIPTION

A system and method for generating easily usable patient-related medical information is disclosed. The system comprises a healthcare provider transformer application that automatically converts patient medical information in a way that is customizable by the healthcare provider. The transforming software processes the patient-related medical information locally, using a local transformation processing, local artificial intelligence engine, or sends the information after pre-processing to an internet server for further processing to produce easy to understand, reader-appropriate medical information. The generated information is suitable for storage, sharing with other healthcare providers, ancillary personnel and staff involved in patient care, patients, and their caretakers as well as any other individuals involves in the patients care. Additionally, the transformer software can generate instructions from standard medical format into hybrid output that is both human and machine-readable. The transformer software can additionally hash the newly generated information and/or instructions into the blockchain and/or mint non-fungible tokens (NFTs) that contain the instructions and include access to them in the blockchain. The hybrid output includes simple instructions with clear text with or without tables and/or illustrations or clarifications, as well as machine-readable elements such as barcodes, QR codes, encrypted codes, and customizable website/application links that allows interactions with other software, machines or allow access to the blockchain or the data stored online. The system can also include a user software application that can read and process the hybrid output and generate additional patient instructions in different forms and customize alarms, calendar events, reminders, patient compliance logs, and means to communicate with the healthcare provider. The system may also include a dedicated website that can process the data in the hybrid output to further display patient instructions, set reminders, or calendar event invitations through the browsing device operating system and interact with a smart device to set physical device reminders/usage data. The patient may directly or indirectly interact with the hybrid output, the native user application, the dedicated website, or the smart device to properly follow medical instructions, medications or eye drop use and log patient usage. Furthermore, the application and/or the website can gamify the usage of medications such as via rewarding the patient points, stars, and rank usage.

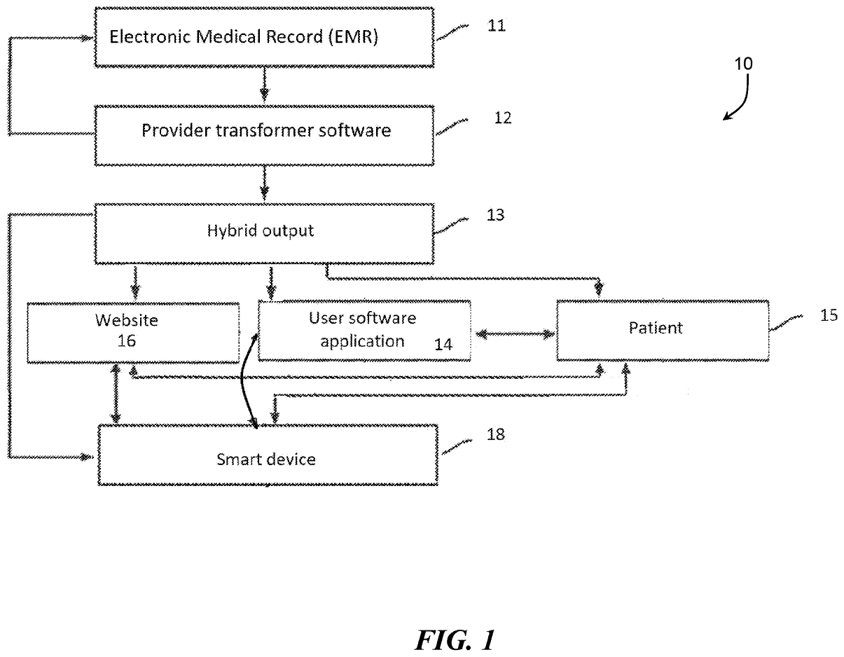

FIG. 1 is a block diagram illustrating the components of system 10 for generating patient-related medical information according to an embodiment of the present invention. As shown in FIG. 1, system 10 includes a healthcare provider transformer application 12, that can process medical instructions text provided for example from electronic medical records 11, a user software application 14, and/or a dedicated website 16.

The healthcare provider transformer application 12 converts patient medical instructions from their medical format into an hybrid output 13 that is both human-readable, such as by the patient 15, and machine-readable. The hybrid output includes layman instructions with clear text with or without tables and/or illustrations, as well as machine-readable features such as barcodes, QR codes, encrypted codes, and customizable website links.

The system 10 may also include a smart device 18, such as a medication or eyedrop holder or dispenser, that can interact with the user software application 14 or the dedicated website 16 via WIFI, Bluetooth, or RF to set physical device reminders/usage data.

In operation, the healthcare provider transformer application 12 converts patient medical instructions in their medical format (for example Xalatan QHS OU) into an hybrid output that is both patient and machine-readable. The hybrid output includes layman instructions with clear text+/−tables and/or illustrations, as well as machine-readable features such as barcodes, QR codes, encrypted codes, and customizable website links. The patient's instruction may specify for example medication name such as latanoprost drop, identifying features such as “teal colored cap”, frequency such as once at bedtime, and method of administration for example one drop in each eye. It may also specify certain common side effects of the medication, reference some instructions related to medication care e.g., to refrigerate it until the time of use, and may reference coupon codes or what to do in the event of a side effect or allergic reaction. The hybrid output is screen displayable, printable, and electronically transferable so that it can be further reviewed by the patient and/or processed by applications, websites, or devices.

The user software application 14 or the dedicated website 16 can then process the hybrid output to generate additional patient instructions in different forms, such as text, tables, photos, figures, references, or more info related to the instructions or medications. The healthcare provider can paste the hybrid output back into the EMR or file the printout back into the patient folder. The user software application 14 may also generate large, high-contrast text or text-to-speech output for users with visual impairments, customize alarms and reminders, and track patient compliance on reading certain data on the hybrid output such as a QR code or an encrypted code.

The patient may interact with the hybrid output, the native application 14, the dedicated website 16, or the smart device 18 to properly follow medical instructions and medications/eye drop use. This can improve patient adherence to medications and avoid obstacles such as forgetting to use the medications or using them incorrectly.

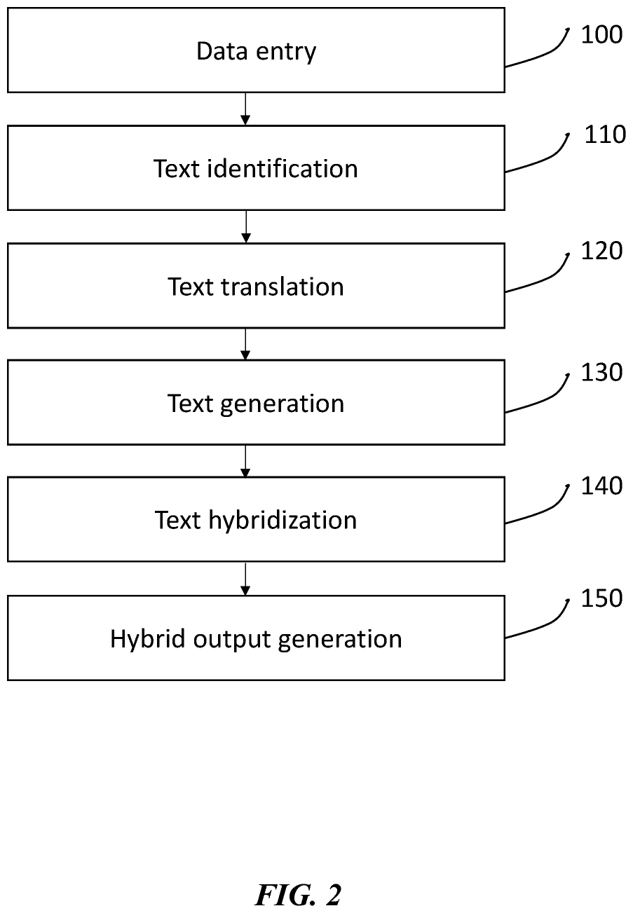

FIG. 2 is a flowchart of the processes by which the provider transformer software converts the provider input into the hybrid output 13.

The process starts with data entry 100 by the healthcare provider into the provider transformer software. This might involve typing, dictation, copy-pasting from or parsing the electronic medical record. Once the data in the medical text is entered and received into the provider transformer software 12, the text undergoes the text identification step 110 where the software identifies the style of medical writing. For example, a medication such as Xalatan, a prescription eye drop used for glaucoma treatment may be entered as Xalatan qhs OU meaning to use Xalatan once at bedtime in each eye. But similar instructions may be written as Xalatan qpm OU (Xalatan once in the afternoon/evening in each eye) or Xalatan 1:1 meaning Xalatan once in each eye. Additionally, the medication may be referred to by its brand name Xalatan, its generic/scientific name Latanoprost, or commonly abbreviated versions such as latano. The function of step 110 is to identify the style and based on the style used for each medication, an appropriate translation algorithm will be applied next. Text that does not follow any known style can trigger an error code to warn the provider to review the text entered for accuracy or advise to use the preferred format. Once the translation algorithm is selected based on step 110, the text translation step 120 takes place where the medication names are converted into the appropriate (for example scientific name) correcting typos, replacing abbreviations with full names. It also translates the frequency of for example “qhs” to “once at bedtime” and translates how to use the medication into simple text such as “OU” becoming “in both eyes”. In step 130 (text generation) takes place via combining the different pieces of the medication name, frequency, mode of use etc. into legible sentences removing extra words, special characters, and spaces. Once the legible simple text is generated, text hybridization 140 is performed by the application added extra separators, formatting, additional text such as information about the medications, their characteristic features such as pill shape/color, eye drop cap color etc. and add general advice, disclaimer, date and time or any other data needed. Additional logos, images, illustrations, tables, as well as the machine-readable barcode, QR code, encrypted code gets added by the software. Step 150 output generation is adjustable by the healthcare provider who can choose using the application user interface the output format and what is included in the output generated.

For example, when the data entry is “Xalatan qhs OU, brimonidine bid OU, dorzolamide/timolol bid OU”, the hybrid output can be something like:

Some other features of the application include functions such as decoding the impression and plan section of the patient chart. Like the process involved in generating medical instructions, the process starts with data entry.

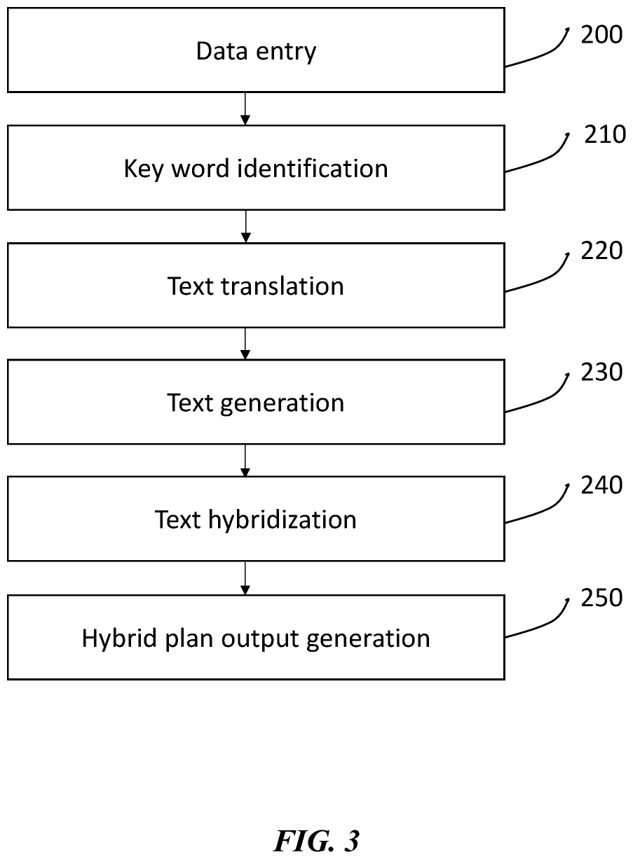

FIG. 3 is a flowchart of the processes by which the provider transformer software converts the provider's instruction and plan into the hybrid output instruction and plan 13.

The process starts with data entry 200 by the healthcare provider into the provider transformer software. This might involve typing, dictation, copy-pasting from or parsing the electronic medical record. Once the impression and plan data in medical text is entered and received into the provider transformer software 12, the text undergoes the keyword identification step 210 where the software identifies the key medical terms and abbreviations. For example, an entered paragraph that says “Severe POAG OU: unacceptable IOP OU, RTC in 2 months for HVF”, which means that the patient has Primary open angle glaucoma in both eyes and that the intraocular pressure is acceptable in each eye and needs to return to the clinic in 2 months for Humphrey visual field”. The keywords are (POAG, OU, IOP, RTC and HVF). To the patient, such terms might be cryptic and deciphering them might be a lengthy process. The software identifies these terms and creates a list of them eliminating the duplications. In step 220, the text translation involves translating the medical term such as POAG into pre-defined simple language definition such as “POAG: primary open angle glaucoma, a type of glaucoma (a group of eye conditions that can cause vision loss) in which the angle between the iris and cornea (the clear front part of the eye) is open, but pressure in the eye is increased, leading to vision loss.” Then moves on to the next term and so on. In step 230 (text generation) takes place via combining the different definitions of medical terms and removing extra words, r characters, and spaces. Once the legible simple text is generated, text hybridization 240 is performed by the application added extra separators, formatting, additional text, etc., and add general advice, disclaimer, date and time, or any other data needed. Additional logos, images, illustrations, tables, as well as the machine-readable barcode, QR code, and encrypted code gets added by the software. Step 250 hybrid plan output generation is adjustable by the healthcare provider who can choose using the application user interface the output format and what is included in the output generated. For example, in the example paragraph above the output could be something like:

“Here are some medical terms from your report today and their explanations:
POAG: primary open angle glaucoma, a type of glaucoma (a group of eye conditions that can cause vision loss) in which the angle between the iris and cornea (the clear front part of the eye) is open, but pressure in the eye is increased, leading to vision loss.
OU: oculus uterque (meaning both eyes), both eyes.
IOP: intraocular pressure, the pressure inside the eye.
RTC: Return to clinic
HVF: peripheral vision or side vision test.”

In certain embodiments, Artificial Intelligence (AI can be used to perform the steps of identification, translation, text generation, hybridization, and output generation

Artificial intelligence (AI) is a rapidly growing field that has the potential to transform industries, including healthcare. One potential use of AI in healthcare is to improve patient care. For example, AI algorithms can be used to analyze electronic health records, identify patterns, and make recommendations for treatment. AI can also be used to assist in the diagnosis of medical images, such as CT scans or X-rays, which can help to improve the accuracy and speed of diagnosis. One of the limitations of the use of AI in processing healthcare records is the poor tolerance to any potential errors made by the AI algorithm.

Natural language processing (NLP) is a subfield of artificial intelligence that focuses on the interaction between humans and computers using natural language. In healthcare, NLP can make it easier for diverse groups of people to understand medical information by processing technical language and converting it into a more accessible language. NLP can also be used to extract valuable information from medical records, such as diagnoses, treatment plans, and test results, which can be useful for population health management and clinical decision support. The use of NLP in healthcare has the potential to improve communication and understanding within the healthcare system, leading to better patient outcomes.

The use of internet-based artificial intelligence (AI) to simplify medical language has the potential to improve communication and understanding within the healthcare system, but there are potential downsides to consider. These include confidentiality concerns related to transferring patient medical information online, the potential for the widespread use of specialty, subspecialty, or provider-specific abbreviations that may not be recognized by the AI model, and the time and knowledge required for healthcare providers to set parameters to optimize the AI model. To address these issues, an intermediary portal that simplifies and optimizes the process of healthcare providers interacting with AI models is needed. It is also important to de-identify medical information before processing by the AI to protect patient privacy.

De-identifying medical records is a challenging task for AI and NLP because it requires a high level of accuracy and attention to detail to effectively remove all personally identifiable information from the records while still preserving the integrity of the medical information. This task can be particularly challenging because of the complex and technical language used in medical records, as well as the need to account for various forms of implicit and explicit identification, such as dates and geographic information. It is also exceptionally challenging for AI to recognize human names that have meaning like “King” or “Can” as a person's name or distinguish it from a name of a device or a procedure named after a person like “Ahmed valve”. Additionally, AI and NLP systems may struggle with understanding the context of the information in the medical records, making it difficult to determine which information is truly identifying and which is not.

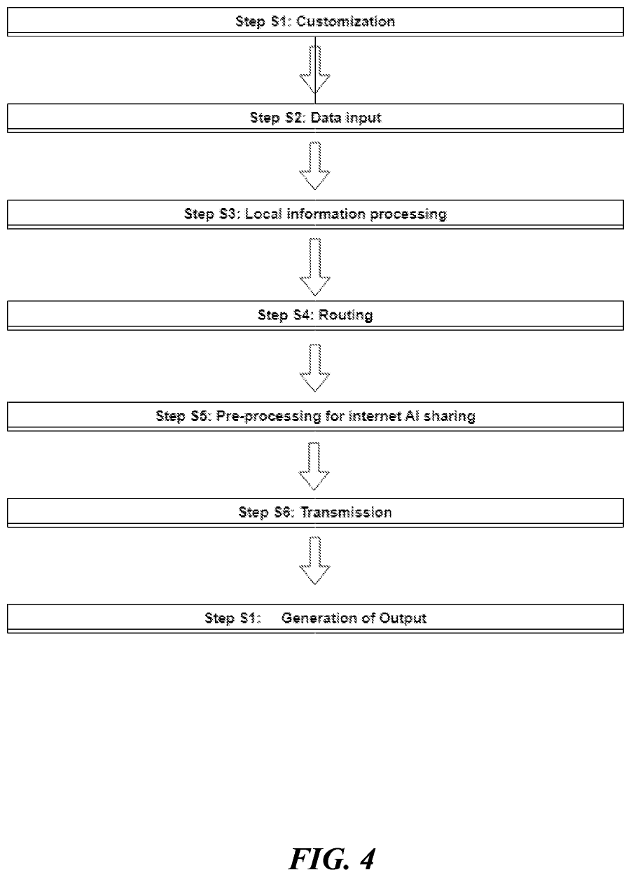

FIG. 4 is a diagram further illustrating the process of the healthcare provider transformer application in accordance with one embodiment. In step S1, the health care provider performs user-friendly optional optimization of the provider transformer application to match their needs. For example, setting preference for local plain text processing, local AI processing or over-the-internet AI processing. Other specialty dictionaries (general, ophthalmology, urology etc.) allow the transformer software to properly translate the abbreviations in the medical text. settings such as creating templates for different ways of processing medical information to be suitable for sharing with different recipients. The transformer application applies these customization parameters as standard presets, for example, “Letter to specialist” which will allow the use of more detailed and technical language, “Letter to primary care or general practitioner” which will be less structured, less technical, and more focused on pertinent info to a non-specialist. A “Letter to patient or their caretakers” is the least technical, more layman term based and illustrated with diagrams and figures if needed. Additionally, templates to create certain forms such as progress forms for nursing homes, insurance companies, and disability can be generated based on the patient notes. In step S2, the healthcare provider inputs patient medical data through typing, copy-pasting from electronic medical records, dictation, or other means. Alternatively, when entry is requested, the transformer application can pull the data directly from the electronic medical records. Step S3 involves local information processing the input information locally on the healthcare provider's computer using common computational techniques such as parsing, replacement, translation, formatting, and re-formatting to de-identify the data, optimize the results of AI processing, or using a local customizable AI software module to perform these tasks in an automated or semi-automated way. The local AI software module is customizable and trainable. In step S4, routing the processed information is either shared directly with the intended audience or sent to an internet AI server for further processing using AI software engine if selected by the healthcare provider. Step S5 is the pre-processing step of the information locally before submitting it to the AI internet server, including de-identifying the patient in a HIPAA-compliant way, formatting the information, and translating abbreviations or terms to the extent needed to match the ultimate recipient. The process of de-identification performed by the provider transformer application is a multi-layer deidentification process involving a multi-step approach. First, the application obtains confidential patient information, such as name, date of birth, social security number, medical record number, address, healthcare providers' names, and insurance providers, directly or indirectly from electronic medical records (EMR). Direct acquisition includes utilization of API protocols to communicate with the EMR or simulating computer keystrokes and mouse movements and clicks to obtain such private data from EMR. This can further involve obtaining private information from certain EMR pages, parsing, optical character recognition of screen captures, utilizing smart phrases, smart texts, dot phrases, or other methods known to pull data from specific data fields of the EMR that store such data. Indirect acquisition can include the provider obtaining these data from the EMR and entering data into the provider transformer application. The provider transformer application searches the medical note to be deidentified for the phrases listed in the chart-specific private information and replace them with generic words or symbols such as “NAME”, “ADDRESS” or “******”. Then other generic deidentification algorithms using natural language processing (NLP) techniques to automatically identify and redact sensitive information. It can also apply machine learning algorithms to predict sensitive information based on patterns in the data and then remove or replace that information. Additionally using certain formatting or letter-case patterns can be used to identify and redact certain pieces of information such as dates and phone numbers. This multi-layer and multi-step approach ensures a high degree of accuracy in removing personally identifiable information from the records while preserving the integrity of the medical information. The deidentified phrases are stored as computer variables that can be used to re-identify the note once it returns back to the Transformer software after processing by the AI internet server.

In step S6, Transmission of customization parameters set by the healthcare provider, are combined with the raw or processed patient medical information and submitted to the AI software via secure ways such as API or secure email. The last step, step S7, is the generation of a sharable output that is customized to the needs of the healthcare provider and suitable for sharing with other healthcare providers, ancillary personnel, and patients. In this step, the transformed software can add custom data to the transformed information provided back by the AI internet server for example, adding back identifying patient info such as patient name, date of birth, health providers' contact info and signature.

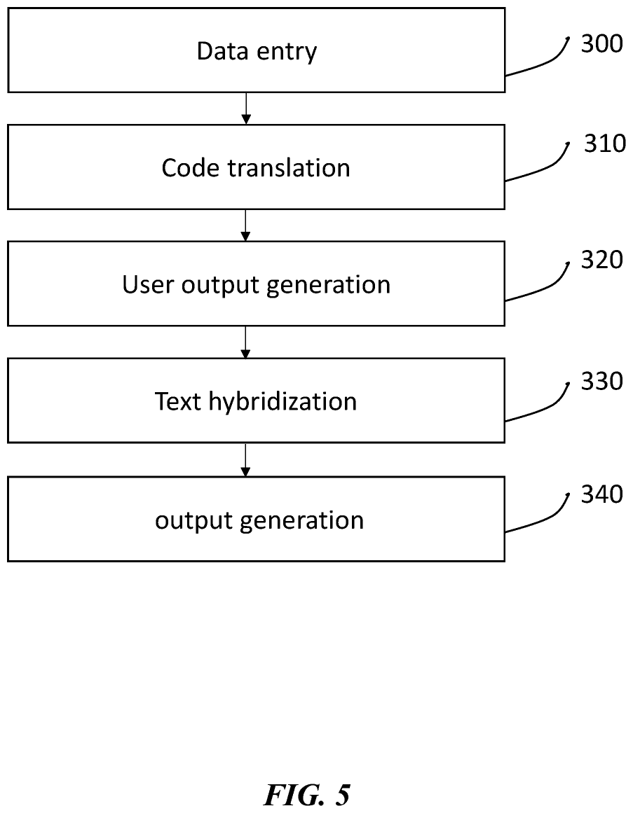

FIG. 5 is a flowchart of the processes by which the user software 14 or dedicated website 16 converts the machine-readable codes on the hybrid output into simple smart patient instruction and plan translation.

The process starts with data entry 300 by the patient into the user software 14 or dedicated website 16. This might involve typing, QR code scanning, copy-pasting from electronically transmitted info such as email or directly injected data from the health care provider or EMR into the user software. Once the data is entered into the software 14 or website 16, the text code, bar or QR code undergoes translation step 310, the code translation involves translating the encrypted code such as a QR code into simple text similar to the simple text on the hybrid output of the medical instructions and translated impression and plan. This allows the user to save an electronic copy of the instruction, make copies, and share them with other healthcare providers or caretakers. In step 320 (user output generation) takes place via displaying the legible plain text into the screen of the used device. Text hybridization 330 is performed by the application adding extra forms of output such as separators, formatting the text in a more visible way such as magnified, high contrast, converting it into speech for the visually impaired or dyslexic patients, creating reminders and alarms based on the instructions. Step 340 output generation is adjustable by the user who can choose using the application user interface the output format and what is included in the output generated.

The system and method of the present invention may have several advantages over prior art systems. For example, by converting patient medical instructions into an hybrid output that is both patient and machine-readable, the system allows healthcare providers to simply convert the instructions from medical jargon that typically exists in patient paper or electronic medical records easily into patient-friendly instructions that present information in a clear and understandable format for patients and their family members, while also providing machine-readable features such as barcodes, QR codes, encrypted codes, and customizable website links that can be processed by the user software application, the dedicated website, or the smart device. Furthermore, the current invention around the secure transfer of instructions to the patient directly from the provider's computer monitor or printer without utilizing a central or a cloud-based server that might be hacked or inaccessible. Furthermore, the system is independent of the EMR used, the operating system used by the healthcare provider, and does not need the patient to even have internet access to utilize the hybrid output or scan it using their smart devices.

The user software application 14 or dedicated website 16 can then generate additional patient instructions in different forms, such as text, tables, photos, figures, references, web links, or more info related to the instructions or medications, which can be customized for users with visual impairments or other needs. The application 14 or dedicated website 16 can also generate alarms and reminders, track patient compliance for example with a log, and provide means for communication with the healthcare provider.

The user software application 14 or dedicated website 16 can also set reminders through the browsing device operating system and interact with a smart device to set physical device reminders and collect usage data.

By interacting with the hybrid output, the native application on a smartphone, smartwatch, tablet, computer, or the dedicated website, or the smart device, patients can properly follow their medical advice and medications/eye drop instructions, which can improve patient adherence to medications and avoid obstacles such as forgetting to use the medications or using them incorrectly.

In certain embodiments, the hybrid instruction output can be generated using a blockchain.

Blockchain is a digital technology that uses cryptography to record and verify transactions and information across a decentralized network of computers. Each transaction is recorded as a “block” and linked to the previous block, creating a “chain” of blocks. This creates a secure and transparent system for tracking and verifying transactions, which has a variety of potential applications, including managing healthcare records.

Non-fungible token (NFT) is a type of cryptocurrency that represents ownership of a unique digital asset such as an image. NFTs are often used to represent digital art, collectibles, or other unique digital items. One of the key features of NFTs is that they are non-fungible, which means that they cannot be exchanged for other assets on a one-to-one basis. The use of blockchain technology allows for a secure and transparent way to track and verify ownership of NFTs.

Blockchain, NFTs, and smart devices have the potential to improve patient education and make it easier for patients to follow medical instructions. Blockchain can be used to securely store and track medical records, while NFTs can represent and track ownership of digital educational materials. Smart devices, such as smartphones and wearable devices, can provide real-time access to medical instructions and alerts. By using these technologies, healthcare providers can provide more personalized, convenient, and effective patient education, improving health outcomes and empowering patients to manage their own health.

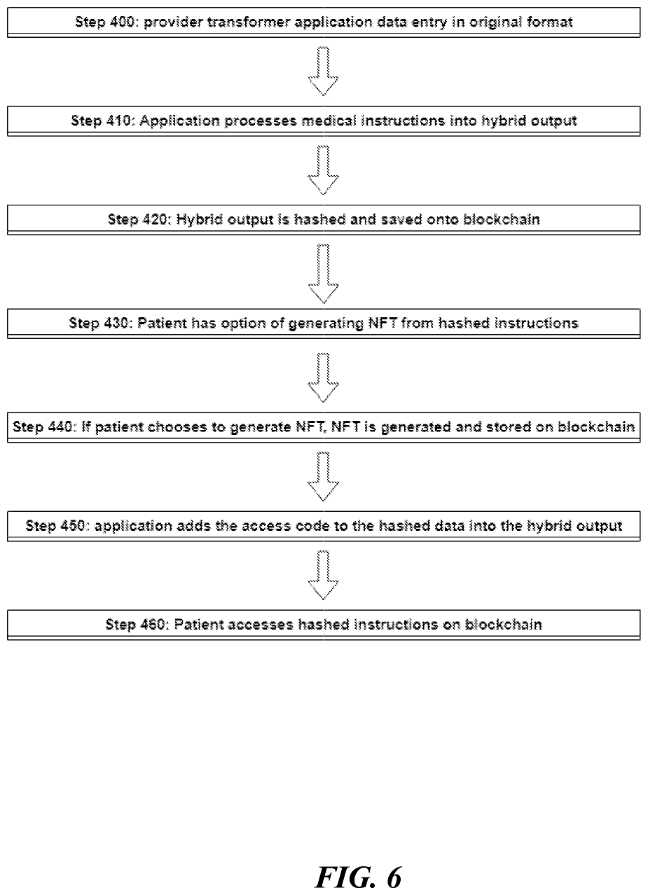

FIG. 6 is a flowchart illustrating the process of hashing the hybrid output 20 onto the blockchain 14 according to an embodiment of the present invention.

The system for hashing easily usable smart patient instructions onto a blockchain includes a healthcare provider transformer application and a blockchain. The process for hashing the instructions onto the blockchain includes the following steps:

Step 400: The healthcare provider transformer application receives the patient's medical instructions in their original format from a healthcare provider. The medical instructions may include text, tables, figures, and other information related to the patient's treatment and medications.

Step 410: The healthcare provider transformer application processes the medical instructions and converts them into a hybrid output. This may involve analyzing the instructions to identify the allowed styles of instructions, parsing the text, identifying keywords, correcting typos, and translating the instructions into different formats that are easily interpreted by the patient. The hybrid output is both patient and machine-readable, with layman's instructions that are easily understandable by the patient and machine-readable features such as barcodes, QR codes, encrypted codes, and customizable website links.

Step 420: The hybrid output is hashed using a cryptographic hash function and saved onto the blockchain. The hashed instructions are stored on the blockchain, allowing for secure and verifiable storage of the hybrid output.

Step 430: The patient has the option of generating a non-fungible token (NFT) from the hashed instructions. If the patient chooses to generate an NFT, the process continues to the next step.

Step 440: The NFT(s) are generated from the hashed instructions and stored on the blockchain. The NFT(s) provides an additional layer of security and verifiability for the instructions. Multiple NFTs that might encrypt the patient's medical instructions, provider data such as their address, website, contact information, and links to say the user application can also be generated as separate NFT(s) or be part of the same NFT.

Step 450: The healthcare provider transformer application adds an access code, links and/or thumbnails to the hashed data and/or NFT and includes it in the hybrid output. This access code allows the patient to access the hashed instructions on the blockchain.

Step 460: The patient accesses the hashed instructions on the blockchain using the access code provided in the hybrid output. The patient can then use the instructions to properly follow medical instructions and medications/eye drop use, improving patient adherence to medications and avoiding obstacles such as forgetting to use the medications or using them incorrectly.

In this way, the present invention provides a system and method for hashing easily usable smart patient instructions onto a blockchain. The hybrid output is converted into a format that is both patient and machine-readable, allowing for easy access and use by the patient. The hashed instructions are stored on the blockchain, providing secure and verifiable storage. The system also allows for the option of generating an NFT from the hashed instructions, providing an additional layer of security and verifiability. The access code included in the hybrid output allows the patient to easily access the hashed instructions on the blockchain and use them to properly follow their medical treatment.

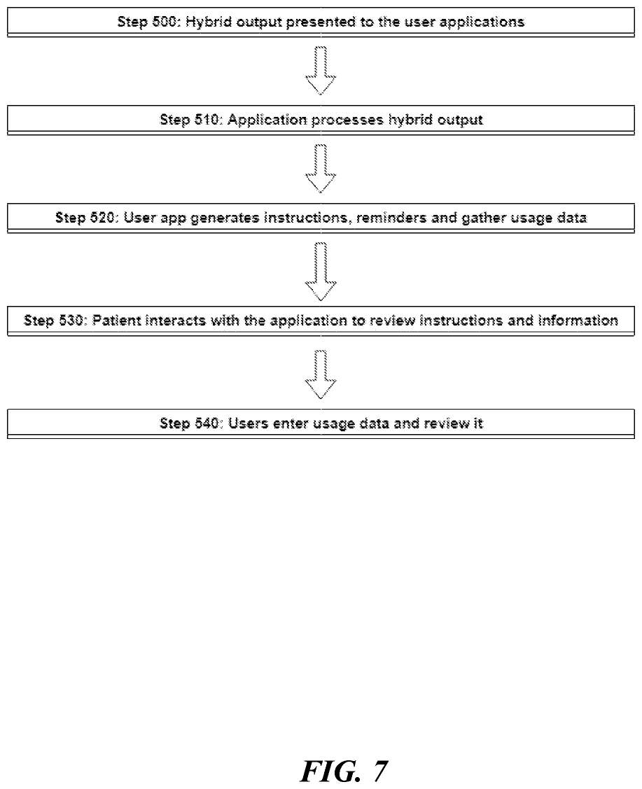

FIG. 7 shows a flow chart demonstrating the steps involved in processing the patient instructions of the hybrid output by the user application and presenting them to the patient, as well as the various features and functionality of the application. In certain embodiments, the dedicated website 16 may perform these steps or provide this functionality instead of or in addition to the user application.

At the top of the flow chart, the hybrid patient instructions are received by the application via camera scanning of the hybrid output elements such as a QR code or manually entered by the patient or transmitted from the provider transformer application. These instructions are then processed to extract and interpret the relevant information, including the prescribed medications and dosages, frequency and method of administration, and any relevant reference materials or patient education resources.

Next, the instructions are presented to the patient in a clear and easy-to-understand format, using text, tables, illustrations, or other visual aids as needed. The patient can then review the instructions and reference materials to understand their treatment plan and medications.

The application also includes several features to set reminders and alarms, alerting the patient when it is time to take their medications. These reminders may be triggered based on the time of day, the patient's location, or other factors, and may be delivered via the application's user interface or via external means such as push notifications or emails. The hybrid output is capable of generating the reminders, calendar events, and instructions with minimal intervention from the patient which minimizes user errors. The patient can customize their reminders if needed, setting them to repeat at regular intervals or adjusting their frequency and timing.

To track the patient's medication usage and adherence, the application includes a log function that records the dates and times that medications were taken. This log may be automatically populated based on the patient's input, or the patient may manually enter their medication usage into the log or when a medication is photographed, or its QR code scanned. The log may also include additional features such as alerts for missed doses or alerts to refill prescriptions when needed.

Finally, the application 14 or dedicated website 16 may include additional features such as gamification techniques to motivate the patient to take their medications as prescribed, or tools for communicating with their healthcare provider. These features are designed to help the patient follow their medical instructions and improve their adherence to their treatment plan.

Gamification of healthcare refers to the use of game design elements and mechanics in healthcare settings to engage and motivate people to adopt healthy behaviors and improve their health outcomes. This applies to patients actively engaging in their healthcare as well as providers gamifying their sharing of medical information and patient education. This can be done with various gamification techniques, such as points, badges, leaderboards, and other game-like elements.

Gamification of healthcare involves using digital technologies, such as mobile apps or wearable devices, to track and monitor health behaviors and provide feedback to users in a game-like format. This can be useful for addressing a wide range of health conditions and behaviors and can be particularly effective at engaging and motivating people who may be less inclined to participate in traditional health interventions.

As part of the gamification strategies, the application 14 or website 16 can utilize the following techniques:
Reminders and notifications-Sending timely reminders and notifications to users is a very effective way to improve adherence to medication regimens.
Personalized feedback-Providing personalized feedback to users based on their medication usage patterns can help to identify any potential issues and offer suggestions for improvement.
Gamified tasks and challenges-Gamified tasks and challenges can provide a sense of purpose and motivation for users to take their medications such as eye drops consistently.
Virtual rewards or incentives-Offering virtual rewards or incentives for medication usage can provide additional motivation for users to take their medications as prescribed.
Social support-Connecting users with others who are also using medications can provide a sense of community and support, which can be helpful for improving adherence.
Points and rewards-Awarding points or other virtual rewards for medication usage can provide an element of motivation for users.
Progress tracking-Tracking users' progress towards their medication usage goals and displaying this progress visually can provide a sense of accomplishment and encourage users to continue taking their medication consistently.
Social comparison-Including a leaderboard or other social comparison feature can provide an element of competition and motivate users to take their medications and/or eye drops more consistently.
Gamified education-Incorporating gamified education content can make learning about medications and/or eye drops usage more engaging and memorable for users.

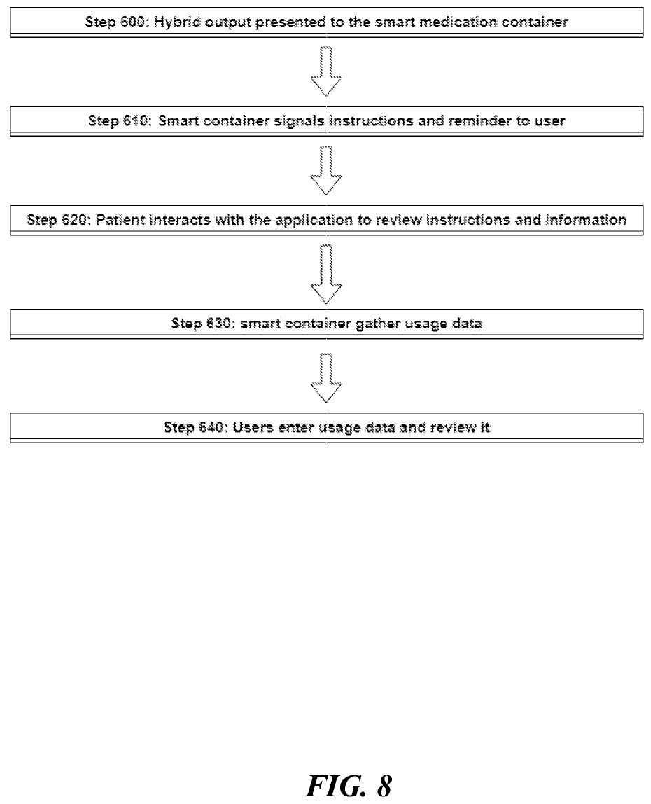

FIG. 8 demonstrates a flow chart that shows the various features and functionality of the smart container, as well as the steps involved in storing, tracking, and administering medications using the container.

The user stores their medications in the designated compartments of the smart container. Each compartment is equipped with a unique human-readable identifier such as color, color LED, number, LCD displaying an identifier, and a machine-readable identifier, such as a barcode or QR code. Additionally, the smart container can be equipped with means to sense its contents via weight, color, and computer vision.

The smart container is configured to process the hybrid output (step 600) directly or via communication with smart devices, such as smartphones or smartwatches, allowing it to send and receive information about the medications based on the hybrid output data stored within it. This includes setting and receiving reminders and other notifications related to the medications.

To assist the user in administering their medications, the smart container includes a number of features such as different illumination colors for each compartment and a display that provides the user with instructions, reminders, and other information (step 610). The container may also include notification methods such as sound, light, vibration, and messages on the display to signal reminders and messages to the user.

In step 630, the user interacts with the different information and signals coming out of the container. The smart container also includes features to open the compartments as needed, allowing the user to easily access their medications. When the user takes their medication, the container may track the date and time of the dose and update its log accordingly. This log may be used to track the user's medication usage and adherence over time.

The smart container is also configured to display instructions and communicate with smart devices, such as smartphones or smart watches (step 640). This allows the container to send and receive information about the medications stored within it, as well as to set and receive reminders and other notifications.

Overall, the smart container provides a convenient and user-friendly tool for storing and administering medications, helping the user to properly follow their medical instructions and improve their adherence to their treatment plan.

The system and method of the present invention may be implemented using a variety of hardware and software components. For example, the healthcare provider transformer application may be implemented as a standalone application running on a healthcare provider computing device such as a computer or server, or as a cloud-based service accessed over the internet. The user software application may be implemented as a native application running on a user computing device such as a smartphone, smartwatch, or tablet, or as a web-based application accessed through a browser. The dedicated website may be implemented as a standalone website accessed through a browser. The smart device may be implemented as a medication or eyedrop holder with built-in wireless connectivity.

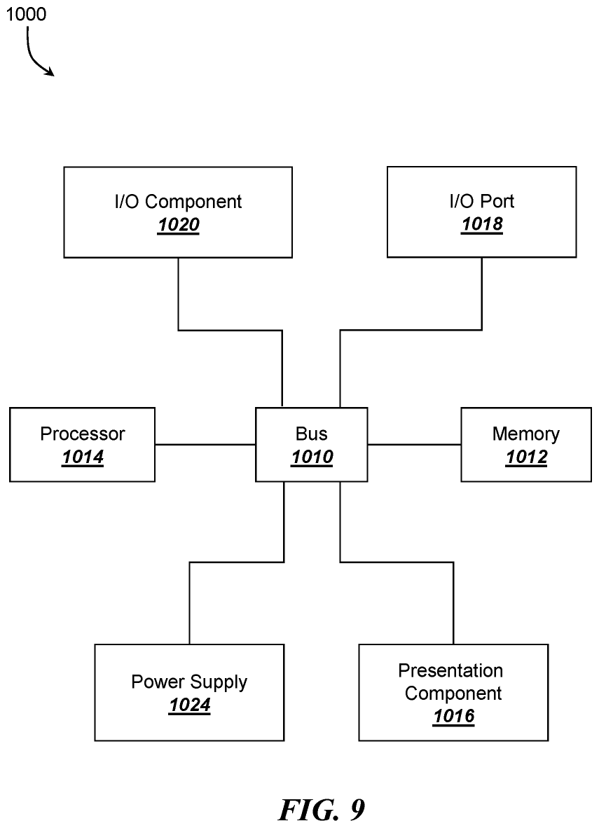

FIG. 9 depicts an example electronic computer, or computing device 1000 that can be used to implement or execute one or more aspects of the present invention, including provider transformation software 12, user software application 14, website 16, and smart device 18. The functionality and hardware of such computing device 1000 may be implemented in any of the electronic hardware systems or subsystems described herein as involving or using a “computer” or “computing device” or the like, or related hardware for providing all or part of the described functionality, provided as a separate device or integrated into a system or subsystem described herein, as would be appreciated and understood by those of skill in the art. The terms “computer”, “computing device”, and the like utilized herein are intended to mean a processor at its most basic form, on up to more complex computing systems, including servers and cloud-based systems, in accordance with conventional meanings of such terms. However, for purpose of completeness, example components and related accessories that are intended to be encompassed by the use of the terms “computer”, “computing device”, “processor”, and the like will be provided below in example nonlimiting form.

The computing device 1000 is merely an illustrative example of a suitable computing environment and in no way limits the scope of the present invention. An “electronic device”, “remote device,” or “personal electronic device” as represented in figures and description herein, can include a “workstation,” a “server,” a “laptop,” a “desktop,” a “hand-held device,” a “mobile device,” a “tablet computer,” a “processor,” or other computing devices, as would be understood by those of skill in the art. Given that the computing device 1000 is depicted for illustrative purposes, embodiments of the present invention may utilize any number of computing devices 1000 in any number of different ways to implement a single embodiment of the present invention. Accordingly, embodiments of the present invention are not limited to a single computing device 1000, as would be appreciated by one with skill in the art, nor are they limited to a single type of implementation or configuration of the example computing device 1000.

The computing device 1000 can include a bus 1010 that can be coupled to one or more of the following illustrative components, directly or indirectly: a memory 1012, one or more processors 1014, one or more presentation components 1016, input/output ports 1018, input/output components 1020, and a power supply 1024. One of skill in the art will appreciate that the bus 1010 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, the figures herein are merely illustrative of an exemplary computing device 1000 that can be used to implement one or more embodiments of the present invention, and in no way limits the invention.

The computing device 1000 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 1000.

The memory 1012 can include computer-storage media in the form of volatile and/or nonvolatile memory. The memory 1012 may be removable, non-removable, or any combination thereof. Exemplary hardware devices are devices such as hard drives, solid-state memory, optical-disc drives, and the like. The computing device 1000 can include one or more processors 1014 that read data from components such as the memory 1012, the various I/O components 1020, etc. Presentation component(s) 1016 present data indications to a user or other device. Exemplary presentation components include a display device, speaker, printing component, vibrating component, etc.

The I/O ports 1018 can enable the electronic or computing device 1000 to be logically coupled to other devices, such as I/O components 1020. Some of the I/O components 1020 can be built into the computing device 1000. Examples of such I/O components 1020 include a sensor (including but not limited to: a camera, optical scanner, RFID scanner, or the like), keypad, touchpad, joystick, recording or storage device, game pad, satellite dish, scanner, printer, wireless device, networking device, and the like, as appropriate.

The system and method of the present invention may be used in a variety of healthcare settings, including hospitals, clinics, pharmacies, and home healthcare settings. It may be particularly useful for patients with complex medical regimens, such as those taking multiple medications or using multiple eye drops, or for patients with visual impairments or other challenges that make it difficult to follow instructions.

It is to be understood that the present invention is not limited to the embodiment described above, and that various changes and modifications may be made without departing from the scope of the invention as defined in the appended claims. For example, the system and method of the present invention may be further enhanced by adding additional features or functionality, such as the ability to adjust the medication reminders based on the patient parameters such as waking up time, bedtime and mealtimes, or the ability to integrate with electronic medical record systems.

The system and method of the present invention may also be configured to allow healthcare providers to customize the hybrid output for each patient, based on the patient's specific needs and medical history. For example, the healthcare provider may be able to select specific medication dosages, frequency of use, or other details to be included in the hybrid output. This can help ensure that patients receive accurate and up-to-date information about their prescribed medications and treatments.

In addition, the system and method of the present invention may include security measures to protect the privacy of patient information. For example, the hybrid output may be encrypted to prevent unauthorized access, and the user software application and dedicated website may require secure login credentials to access patient information. The encrypted information may also be decrypted using one of the patient-specific demographic data or passcode.

The system and method of the present invention may be used in a variety of healthcare settings to improve patient adherence to prescribed medications and treatments. For example, in a hospital setting, the system may be used to provide patients with clear and easily understandable instructions for taking their medications or following other medical orders while they are in the hospital. The system may also be used to track patient compliance with medication regimens and to provide alerts to healthcare providers if a patient is not following their prescribed treatment plan.

In a home healthcare setting, the system may be used to provide patients with instructions for taking their medications or following other medical orders after they have been discharged from the hospital. The system may also be used to track patient compliance with medication regimens and to provide reminders to patients to take their medications or follow other medical orders.

In a pharmacy setting, the system may be used to provide patients with clear and easily understandable instructions for taking their medications and to track patient compliance with medication regimens. The system may also be used to provide alerts to healthcare providers if a patient is not following their prescribed treatment plan.

The foregoing description of the preferred embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A system for generating patient-related medical information, the system comprising:
a healthcare provider transformer application for automatically converting patient medical information into a language suitable for sharing with a variety of individuals involved in a patient's care in a way customizable by the healthcare provider, the healthcare transformer application configured to:
receive medical data inputted by a healthcare provider;
identify key medical terms and abbreviations from the inputted data;
translate the key medical terms and abbreviations into pre-defined simple language definitions;
generate a legible simple text by combining the definitions;
hybridize the simple text by adding one or more of extra separators, formatting, additional text, general advice, disclaimer, date and time, logos, images, illustrations, tables, and a machine-readable barcode, QR code, and encrypted code;
generate a hybrid output based on the hybridized text; and
output the hybrid output wherein the hybrid output comprises instruction that are both human-readable and machine-readable.

2. The system of claim 1, wherein the transformer application processes the patient-related medical information locally, using a local transformation processing, local artificial intelligence engine, or sending the information after pre-processing to an internet server for further processing to produce easy to understand, reader-appropriate medical information.

3. The system of claim 1, wherein the hybrid output further comprises a plan.

4. The system of claim 1, wherein the transformer application hashes generated information and/or instructions into a blockchain and/or mints non-fungible tokens (NFTs) that contain the instructions and include access to the instructions in the blockchain.

5. The system of claim 1, wherein the transformer application is further configured to:
optimize the transformer software to match needs of the healthcare provider, including setting preferences for local plain text processing, local AI processing, or internet AI processing;
select specialty dictionaries for the transformer software;
create templates for different ways of processing medical information;
de-identify data, optimize results of AI processing, or use a local customizable AI software module to perform tasks in an automated or semi-automated way; and
transmit customization parameters combined with raw or processed patient medical information to an AI software via secure ways.

6. The system of claim 1, further comprising a user software application for reading and processing the hybrid output and generating additional patient instructions in different forms and customizing alarms, calendar events, reminders, patient compliance logs, and means to communicate with the healthcare provider the user software application configured to:
receive the hybrid output;
extract and interpret relevant information from the instructions of the hybrid output;
present the instructions to the patient in a clear and easy-to-understand format; and
set reminders and alarms to help the patient follow the instructions.

7. The system of claim 1, further comprising a dedicated website for processing data in the hybrid output to further display patient instructions, set reminders, or calendar event invitations through a browsing device operating system and interact with a smart device to set physical device reminders/usage data, the dedicated website configured to:
receive hybrid output;
extract and interpret relevant information from the instructions of the hybrid output;
present the instructions to the patient in a clear and easy-to-understand format; and
set reminders and alarms to help the patient follow the instructions.

8. The system of claim 1, wherein a patient may directly or indirectly interact with the hybrid output, a user application, a dedicated website, or a smart device to properly follow medical instructions, medications or eye drop use and log patient usage.

9. The system of claim 8, wherein the application and/or the website gamifies the usage of the medications such as via rewarding patient points, stars, and rank usage.

10. A system for generating patient instructions from medical instructions, the system comprising:
a healthcare provider transformer application on a healthcare computing device that receives medical instructions from a healthcare provider in a medical format and converts the medical instructions into an hybrid output;
a user software application on a user computing device that processes the hybrid output to generate additional patient instructions in different forms;
a dedicated website that enables access to the hybrid output;
a smart device that interacts with the user software application or the dedicated website to set physical device reminders and usage data;
wherein the hybrid output includes layman instructions with clear text and/or tables and/or illustrations, and machine-readable features such as barcodes, QR codes, encrypted codes, and customizable website links; and
wherein the hybrid output is screen displayable, printable, and electronically transferable;
wherein the user software application is configured to generate large, high contrast text or text-to-speech output for users with visual impairments, customize alarms and reminders, and track patient compliance on reading the hybrid output.

11. The system according to claim 10, wherein the healthcare provider transformer application includes text identification that identifies a style of medical writing, and a text translation step that converts medication names into an appropriate name, correcting typos, replacing abbreviations with full names, and translating frequency of use.

12. The system according to claim 10, wherein the healthcare provider transformer application includes text generation that combines different pieces of medication name, frequency, mode of use, etc. into legible sentences, and text hybridization step that adds extra separators, formatting, additional text such as information about the medications, logos, images, illustrations, tables, and the machine-readable barcode, QR code, encrypted code.

13. The system according to claim 10, wherein the healthcare provider transformer application includes output generation that is adjustable by the healthcare provider to choose the output format and what is included in the output generated.

14. The system according to claim 10, wherein the healthcare provider transformer application is further configured to decode an impression and plan section of a patient chart.

15. A method for transforming medical data, comprising:
inputting medical data by a healthcare provider into a provider transformer software;
identifying key medical terms and abbreviations from the inputted data;
translating the key medical terms and abbreviations into pre-defined simple language definitions;
generating a legible simple text by combining the definitions;
hybridizing the simple text by adding extra separators, formatting, additional text, general advice, disclaimer, date and time, logos, images, illustrations, tables, and a machine-readable barcode, QR code, or encrypted code;
generating a hybrid output based on the hybridized text; and
outputting the hybrid output.

16. The method of claim 15, further comprising:
optimizing the transformer software to match needs of the healthcare provider, including setting preferences for local plain text processing, local AI processing, or internet AI processing;
selecting specialty dictionaries for the transformer software;
creating templates for different ways of processing medical information;
de-identifying data, optimizing results of AI processing, or using a local customizable AI software module to perform tasks in an automated or semi-automated way; and
transmitting customization parameters combined with raw or processed patient medical information to an AI software via secure ways.

17-18. (canceled)

19. A method for processing hybrid patient instructions, comprising:
a. receiving hybrid patient instructions, via camera scanning of hybrid output elements, such as a QR code, or manually entered by a patient, or transmitted from a provider transformer application;
b. extracting and interpreting relevant information from the hybrid patient instructions, including prescribed medications and dosages, frequency and method of administration, and any relevant reference materials or patient education resources;
c. presenting the instructions to the patient in a clear and easy-to-understand format, using text, tables, illustrations, or other visual aids as needed;
d. setting reminders and alarms, alerting the patient when it is time to take their medications, triggered based on a time of day, the patient's location, or other factors, and delivered via the application's user interface or via external means such as push notifications or emails;
e. tracking the patient's medication usage and adherence, via a log, which may be automatically populated based on the patient's input, or manually entered by the patient;
f. utilizing gamification techniques to motivate the patient to take their medications as prescribed;
g. providing personalized feedback to users based on their medication usage patterns;
h. offering virtual rewards or incentives for medication usage;
i. connecting users with others who are also using medications;
j. awarding points or other virtual rewards for medication usage;
k. tracking users' progress towards their medication usage goals;
l. including a leaderboard or other social comparison feature; and
m. incorporating gamified education content.

20. A system for providing healthcare instructions comprising:
a healthcare provider transformer application configured to receive a medical instruction and generate a hybrid output;
a user software application configured to receive the hybrid output generate instructions based on the hybrid output;
a dedicated website configured to allow a healthcare provider to customize the hybrid output; and
a smart device configured to store the hybrid output and transmit the hybrid output to a user.

21. The system as in claim 20, wherein the healthcare provider transformer application is further configured to receive patient information and generate a medical instruction based on the patient information.

22. The system as in claim 20, wherein the user software application is further configured to receive the hybrid output from the healthcare provider transformer application and generate a user interface for displaying the hybrid output.

23. The system as in claim 20, wherein the smart device is further configured to track a user's medication usage and adherence over time.

24. The system as in claim 20, wherein the smart device includes means to sense its contents via weight, color, and computer vision.

25. The system as in claim 20, wherein the smart device includes different illumination colors for each compartment and a display that provides the user with instructions, reminders, and other information.

26. The system as in claim 20, wherein the smart device includes notification methods such as sound, light, vibration, and messages on a display to signal reminders and messages to the user.

27-53. (canceled)
